(19) United States
(12) Patent Application Publication
(10) Pub. No.: US 2015/0266920 A1
(43) Pub. Date: Sep. 24, 2015

(54) MACROCYCLIC KETOAMIDE IMMUNOPROTEASOME INHIBITORS

(71) Applicant: HOFF-MANN-LA ROCHE INC., Nutley, NJ (US)

(72) Inventors: Stephen M. Lynch, Westfield, NJ (US); Arjun Narayanan, Cambridge, MA (US)

(21) Appl. No.: 14/434,495

(22) PCT Filed: Oct. 9, 2013
(86) PCT No.: PCT/EP2013/070993

§ 371 Date: Apr. 9, 2015

Related U.S. Application Data
(60) Provisional application No. 61/712,840, filed on Oct. 12, 2012.

(51) Int. Cl. C07K 5/12

(52) U.S. Cl. CPC C07K 5/123; A61K 38/00

(57) ABSTRACT

The invention is concerned with the compounds of formula (I):

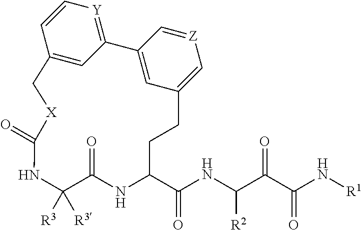

and pharmaceutically acceptable salts thereof, wherein X, Y, Z, R¹, R², R³ and R^{3′} are defined in the detailed description and claims. In addition, the present invention relates to methods of manufacturing and using the compounds of formula I as well as pharmaceutical compositions containing such compounds. The compounds of formula I are LMP7 inhibitors and may be useful in treating associated inflammatory diseases and disorders such as, for example, rheumatoid arthritis, lupus and irritable bowel disease.

### FIELD OF THE INVENTION

The present invention relates to organic compounds useful for therapy and/or prophylaxis in a mammal of an inflammatory disease or disorder, and in particular to macrocyclic ketoamide compounds for the treatment of rheumatoid arthritis, lupus and irritable bowel disease (IBD), their manufacture, pharmaceutical compositions containing them and their use as LMP7 inhibitors.

### BACKGROUND OF THE INVENTION

LMP7 is an essential component of the immunoproteasome, mainly expressed in immune cells such as T/B lymphocytes and monocytes, as well as non-immune cells that have exposed to inflammatory cytokines, including IFN-γ and TNFα. Immunoproteasome plays an essential role in generation of antigenic peptide repertoire and shaping MHC class I restricted CD8+ T cell response. Moebius J. et al. European Journal of Immunology. 2010; Basler, M. et al. Journal of Immunology. 2004. 3925-34. Emerging data suggested that LMP7 also regulate inflammatory cytokine production and immune cell functions beyond the regulation of MHC class I mediated antigen presentation.

A small molecule LMP7 inhibitor, PR-957, has been shown to potently block Th1/17 differentiation, B cell effector functions and production of inflammatory cytokines (ILA TNF-α, IL-23). Muchamuel T et al. Natural Medicine. 2009. 15, 781-787; Basler M. et al. Journal of Immunology. 2010, 634-41.

In addition, LMP7 blockade with PR-957 has been demonstrated to produce therapeutic benefits in several preclinical autoimmune disease models. First, PR-957 was demonstrated to significantly decrease disease score in mouse CAIA and CIA arthritis models, with hallmarks of significantly reduced inflammation and bone erosion. Muchamuel T. et al. Natural Medicine. 2009. 15, 781-787. In addition, PR-957 reduced plasma cells numbers and levels of anti-dsDNA IgG in MRL/lpr lupus-prone mice model, and prevented disease progression in these mice. Ichikawa H T et al. Arthritis & Rheumatism. 2012. 64, 493-503. Furthermore, PR-957 reduced inflammation and tissue destruction in a DSS-induced colitis model in mice. Basler M et al. Journal of immunology. 2010, 634-41. Lastly, LMP7 knockout mice had also been shown to be protected from disease in IBD models. Schmidt N et al. Gut 2010. 896-906.

Taken together, data strongly suggests that LMP7 activity is closely related to the functions of B/T lymphocytes and production of inflammatory cytokines, all of which are clinically validated targets/pathways in the pathogenesis of rheumatoid arthritis, lupus and IBD. Thus, existing data have provided strong rationale for targeting LMP7 for autoimmune disease indications. Due to potential liability with long term usage of a covalent inhibitor in chronic diseases like autoimmunity, a covalent reversible or non-covalent small molecule LMP7 inhibitor is highly desired for autoimmune disease indications.

### SUMMARY OF THE INVENTION

The invention provides for a compound of formula (I):

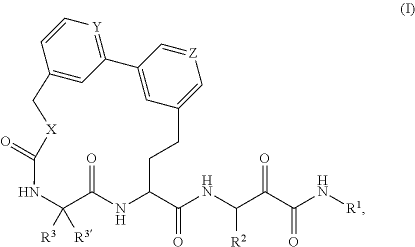

wherein:
X is CH₂, 0 or NH;
Y is CH or N;
Z is CH or N;
R¹ is C₁₋₇ alkyl, C₃₋₈ cycloalkyl or —CH₂-phenyl;
R² is C₁₋₇ alkyl or —CH₂-phenyl; and
one of R³ or R^{3′} is hydrogen and the other is C₃₋₈ cycloalkyl, unsubstituted C₁₋₇ alkyl or C₁₋₇ alkyl substituted with C₁₋₇ alkoxy, or
R³ and R^{3′}, together with the carbon atom to which they are attached, combine to form a C₃₋₈ cycloalkyl moiety,
or a pharmaceutically acceptable salt thereof.

The invention also provides for pharmaceutical compositions comprising the compounds, methods of using the compounds and methods of preparing the compounds.

All documents cited to or relied upon are expressly incorporated herein by reference.

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule. For example, the R variables of formula I refer to moieties that are attached to the core structure of formula I by a covalent bond.

In reference to a particular moiety with one or more hydrogen atoms, the term “substituted” refers to the fact that at least one of the hydrogen atoms of that moiety is replaced by another substituent or moiety. For example, the term “C₁₋₇ alkyl substituted by halogen” refers to the fact that one or more hydrogen atoms of a C₁₋₇ alkyl (as defined below) is replaced by one or more halogen atoms (e.g., trifluoromethyl, difluoromethyl, fluoromethyl, chloromethyl, etc.).

The term “alkyl” refers to an aliphatic straight-chain or branched-chain saturated hydrocarbon moiety having 1 to 20 carbon atoms. In particular embodiments the alkyl has 1 to 10 carbon atoms.

The term “C₁₋₇ alkyl” refers to an alkyl moiety having 1 to 7 carbon atoms. Examples of C₁₋₇ alkyls include methyl, ethyl, propyl, isopropyl, butyl, isobutyl, sec-butyl and tert-butyl.

The term “C₁₋₇ alkoxy” denotes a group of the formula —O—R′, wherein R′ is an alkyl group. Examples of C₁₋₇ alkoxy moieties include methoxy, ethoxy, isopropoxy, and tert-butoxy.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

The terms “halo”, “halogen” and “halide”, which may be used interchangeably, refer to a substituent fluoro, chloro, bromo, or iodo.

“C₃₋₈ cycloalkyl” means a monovalent saturated carbocyclic moiety having mono- or bicyclic rings. The C_{3-g} cycloalkyl moiety can optionally be substituted with one or more substituents. Examples of C₃₋₈ cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

Unless otherwise indicated, the term “hydrogen” or “hydro” refers to the moiety of a hydrogen atom (—H) and not H₂.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. The compounds of the present invention can also be present in the form of pharmaceutically acceptable esters (i.e., the methyl and ethyl esters of the acids of formula I to be used as prodrugs). The compounds of the present invention can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers” and fall within the scope of the invention. Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

In the practice of the method of the present invention, an effective amount of any one of the compounds of this invention or a combination of any of the compounds of this invention or a pharmaceutically acceptable salt or ester thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

In detail, the present invention provides for compounds of formula (I):

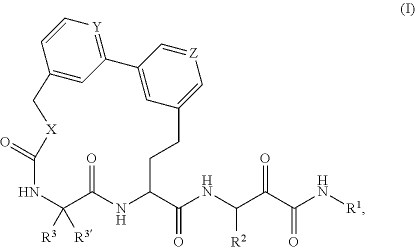

wherein:
X is CH₂, 0 or NH;
Y is CH or N;
Z is CH or N;
R¹ is C₁₋₇ alkyl, C₃₋₈ cycloalkyl or —CH₂-phenyl;
R² is C₁₋₇ alkyl or —CH₂-phenyl; and
one of R³ or R^{3′} is hydrogen and the other is C₃₋₈ cycloalkyl, unsubstituted C₁₋₇ alkyl or C₁₋₇ alkyl substituted with C₁₋₇ alkoxy, or
R³ and R^{3′}, together with the carbon atom to which they are attached, combine to form a C₃₋₈ cycloalkyl moiety,
or a pharmaceutically acceptable salt thereof.

In another embodiment, the invention provides for a compound according to formula (I), wherein X is CH₂.

In another embodiment, the invention provides for a compound according to formula (I), wherein X is O or NH.

In another embodiment, the invention provides for a compound according to formula (I), wherein Y is CH.

In another embodiment, the invention provides for a compound according to formula (I), wherein Y is N.

In another embodiment, the invention provides for a compound according to formula (I), wherein Z is CH.

In another embodiment, the invention provides for a compound according to formula (I), wherein Z is N.

In another embodiment, the invention provides for a compound according to formula (I), wherein X is CH₂, Y and Z are CH.

In another embodiment, the invention provides for a compound according to formula (I), wherein R′ is methyl, —CH₂-phenyl or cyclopropyl.

In another embodiment, the invention provides for a compound according to formula (I), wherein R′ is —CH₂-phenyl.

In another embodiment, the invention provides for a compound according to formula (I), wherein R² is butyl or —CH₂-phenyl.

In another embodiment, the invention provides for a compound according to formula (I), wherein R² is —CH₂-phenyl.

In another embodiment, the invention provides for a compound according to formula (I), wherein one of R³ or R^{3′} is hydrogen and the other is cyclopropyl, methyl, or —CH₂OCH₃.

In another embodiment, the invention provides for a compound according to formula (I), wherein R³ is methyl and R³ is hydrogen.

In another embodiment, the invention provides for a compound according to formula (I), wherein R³ and R^{3′}, together with the carbon atom to which they are attached, combine to form a cyclopropyl moiety.

A particular embodiment of the invention relates to compounds of formula (F)

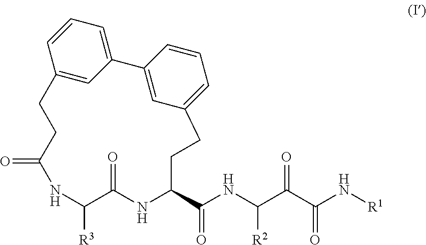

wherein R¹, R² and R³ are as defined above, more particularly R′ is —CH₂-phenyl, R² is —CH₂-phenyl and R³ is methyl.

In another embodiment, the invention provides for a compound according to formula (I), wherein the compound is:

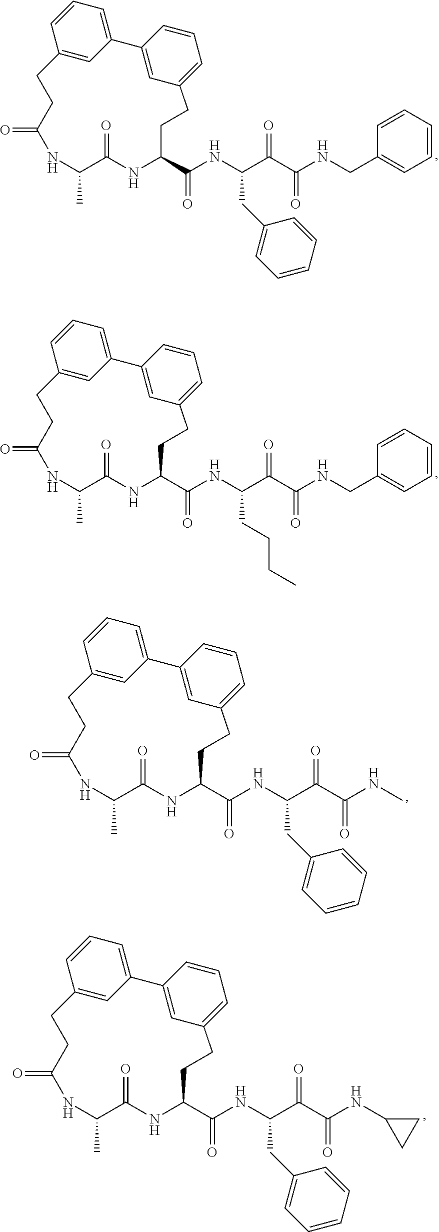 [SMILES: C[C@@H]1NC(=O)CCc2cccc(c2)-c2cccc(c2)CC[C@@H](C(=O)N[C@@H](Cc2ccccc2)C(=O)C(=O)NCc2ccccc2)NC1=O ; CCCC[C@H](NC(=O)[C@@H]1CCc2cccc(c2)-c2cccc(c2)CCC(=O)N[C@@H](C)C(=O)N1)C(=O)C(=O)NCc1ccccc1 ; CNC(=O)C(=O)[C@H](Cc1ccccc1)NC(=O)[C@@H]1CCc2cccc(c2)-c2cccc(c2)CCC(=O)N[C@@H](C)C(=O)N1 ; C[C@@H]1NC(=O)CCc2cccc(c2)-c2cccc(c2)CC[C@@H](C(=O)N[C@@H](Cc2ccccc2)C(=O)C(=O)NC2CC2)NC1=O]

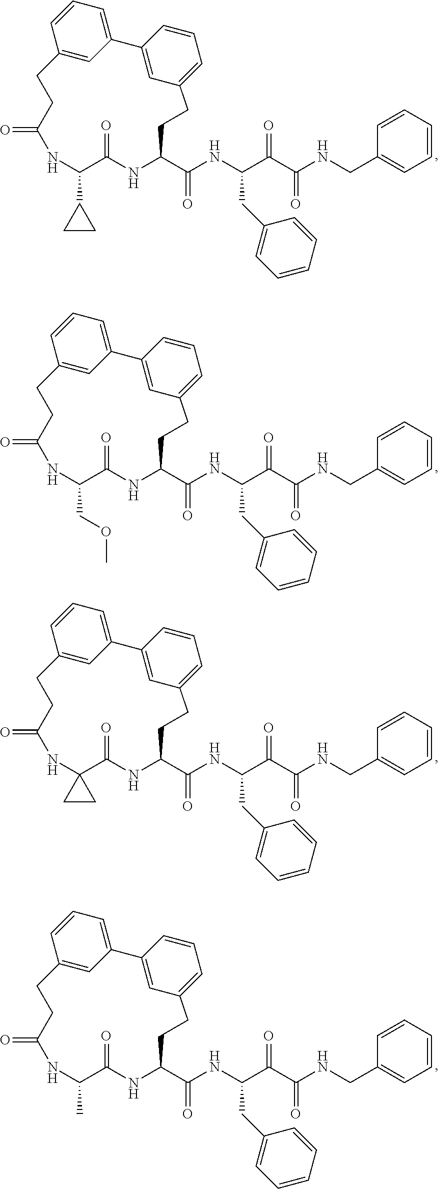 [SMILES: O=C1CCc2cccc(c2)-c2cccc(c2)CC[C@@H](C(=O)N[C@@H](Cc2ccccc2)C(=O)C(=O)NCc2ccccc2)NC(=O)[C@H](C2CC2)N1 ; COC[C@@H]1NC(=O)CCc2cccc(c2)-c2cccc(c2)CC[C@@H](C(=O)N[C@@H](Cc2ccccc2)C(=O)C(=O)NCc2ccccc2)NC1=O ; O=C1CCc2cccc(c2)-c2cccc(c2)CC[C@@H](C(=O)N[C@@H](Cc2ccccc2)C(=O)C(=O)NCc2ccccc2)NC(=O)C2(CC2)N1 ; C[C@@H]1NC(=O)CCc2cccc(c2)-c2cccc(c2)CC[C@@H](C(=O)N[C@@H](Cc2ccccc2)C(=O)C(=O)NCc2ccccc2)NC1=O]

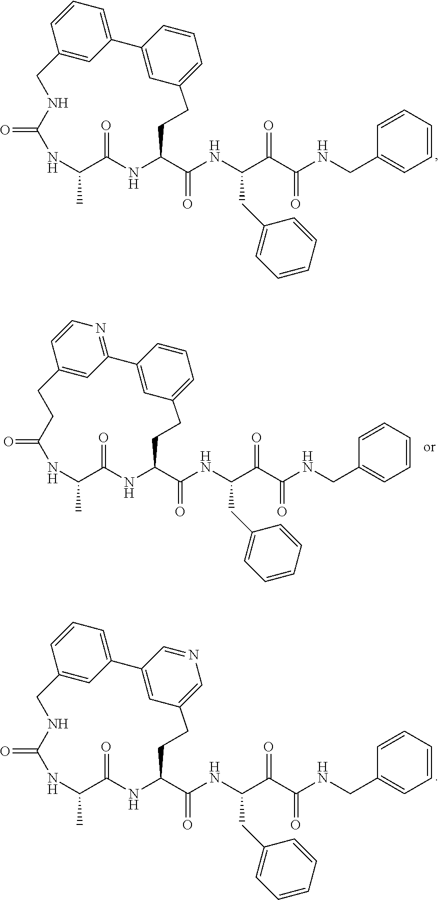 [SMILES: C[C@@H]1NC(=O)NCc2cccc(c2)-c2cccc(c2)CC[C@@H](C(=O)N[C@@H](Cc2ccccc2)C(=O)C(=O)NCc2ccccc2)NC1=O ; C[C@@H]1NC(=O)CCc2ccnc(c2)-c2cccc(c2)CC[C@@H](C(=O)N[C@@H](Cc2ccccc2)C(=O)C(=O)NCc2ccccc2)NC1=O ; C[C@@H]1NC(=O)NCc2cccc(c2)-c2cncc(c2)CC[C@@H](C(=O)N[C@@H](Cc2ccccc2)C(=O)C(=O)NCc2ccccc2)NC1=O]

In another embodiment, the invention provides for a compound according to formula (I), wherein said compound is:

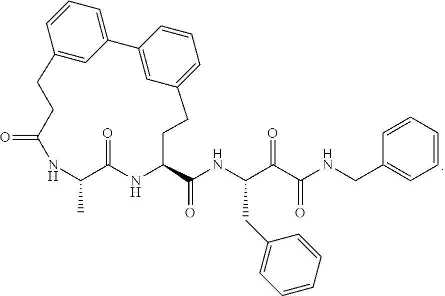 [SMILES: C[C@@H]1NC(=O)CCc2cccc(c2)-c2cccc(c2)CC[C@@H](C(=O)N[C@@H](Cc2ccccc2)C(=O)C(=O)NCc2ccccc2)NC1=O]

In another embodiment, the invention provides for a compound according to formula (I), wherein said compound is (9S,12S)-12-Methyl-11,14-dioxo-10,13-diaza-tricyclo[15.3.1.1^{2,6}]docosa-1(20),2(22),3,5,17(21),18-hexaene-9-carboxylic acid ((S)-1-benzyl-2-benzylcarbamoyl-2-oxoethyl)-amide

In another embodiment, the invention provides for a pharmaceutical composition, comprising a therapeutically effective amount of a compound according to formula (I) and a pharmaceutically acceptable carrier.

In another embodiment, the invention provides for a compound according to formula (I) for use as a therapeutically active substance.

In another embodiment, the invention provides for the use of a compound according to formula (I) for the treatment or prophylaxis of an inflammatory disease or disorder, particularly the inflammatory disease or disorder is selected from rheumatoid arthritis, lupus and irritable bowel disease.

In another embodiment, the invention provides for the use of a compound according to formula (I) for the preparation of a medicament for the treatment or prophylaxis of an inflammatory disease or disorder, particularly the inflammatory disease or disorder is selected from rheumatoid arthritis, lupus and irritable bowel disease.

In another embodiment, the invention provides for a compound according to formula (I) for the treatment or prophylaxis of an inflammatory disease or disorder, particularly the inflammatory disease or disorder is selected from rheumatoid arthritis, lupus and irritable bowel disease.

In another embodiment, the invention provides for a method for treating an inflammatory disease or disorder selected from rheumatoid arthritis, lupus and irritable bowel disease (IBD), comprising the step of administering a therapeutically effective amount of a compound according to formula (I) to a subject in need thereof.

In another embodiment, provided is an invention as hereinbefore described.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Compounds of the invention may be made by any number of conventional means. For example, they may be made according to the processes outlined in Schemes 1 to 4 below.

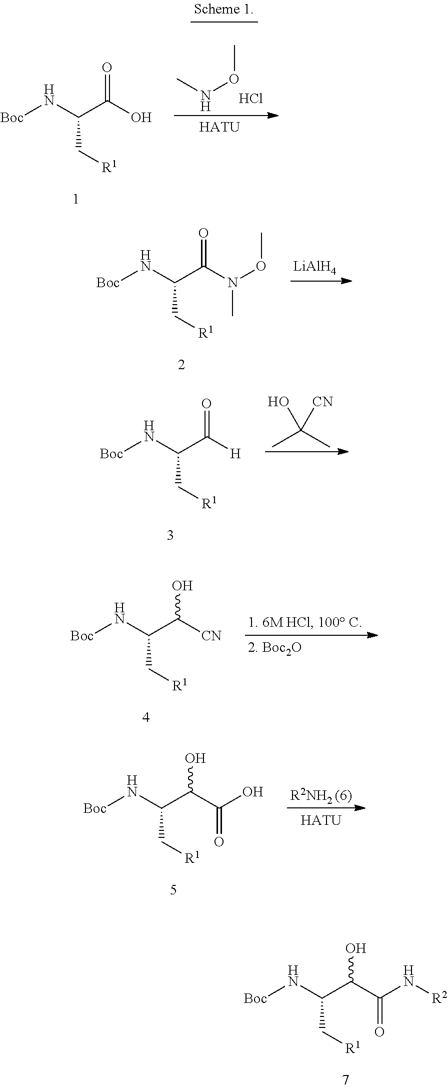

As shown in Scheme 1, the N-Boc protected amino acid 1 can be converted to the Weinreb amide 2 then can be reduced to the aldehyde 3 using lithium aluminum hydride (LiAlH₄). The aldehyde can be immediately treated with acetone cyanohydrin to form the new cyanohydrin 4 as a mixture of diastereomers. Hydrolyzing the nitrile to the carboxylic acid along with loss of the Boc protecting group can be performed by heating with hydrochloric acid. The Boc group can be reinstalled using di-tert-butyl dicarbonate to afford the acid 5 which can be subsequently coupled with an appropriate amine 6 using an activating reagent such as HATU to provide the hydroxyamide 7.

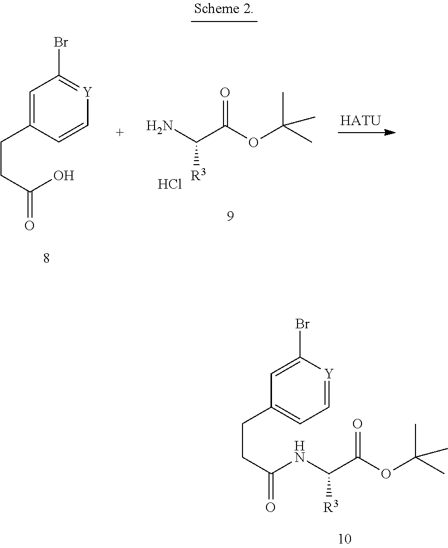

As seen in Scheme 2, the acid 8 can be coupled with an appropriately selected amino acid 9 using an activating reagent such as HATU to afford the amide 10.

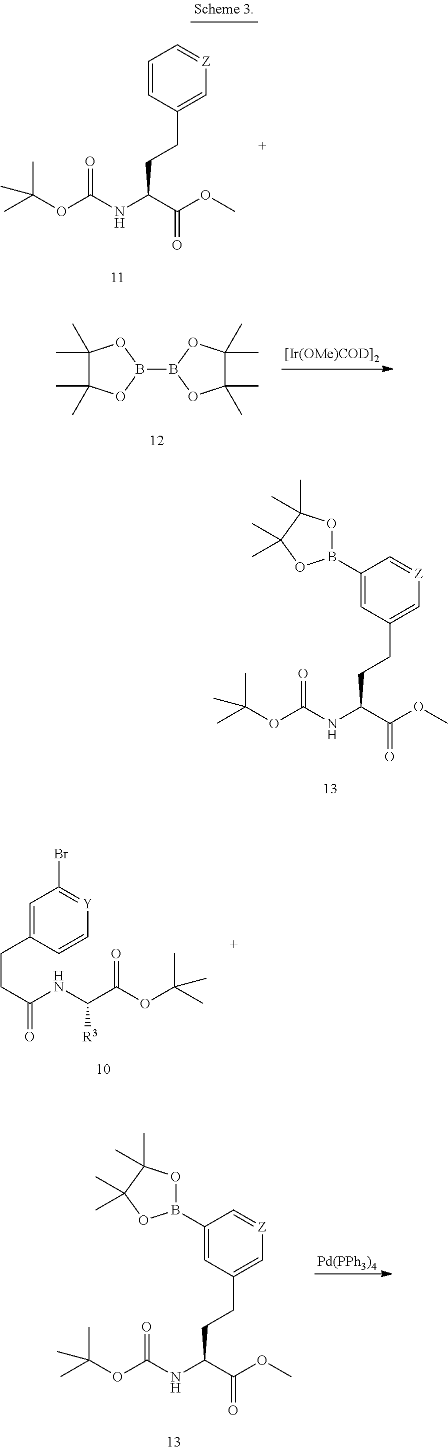

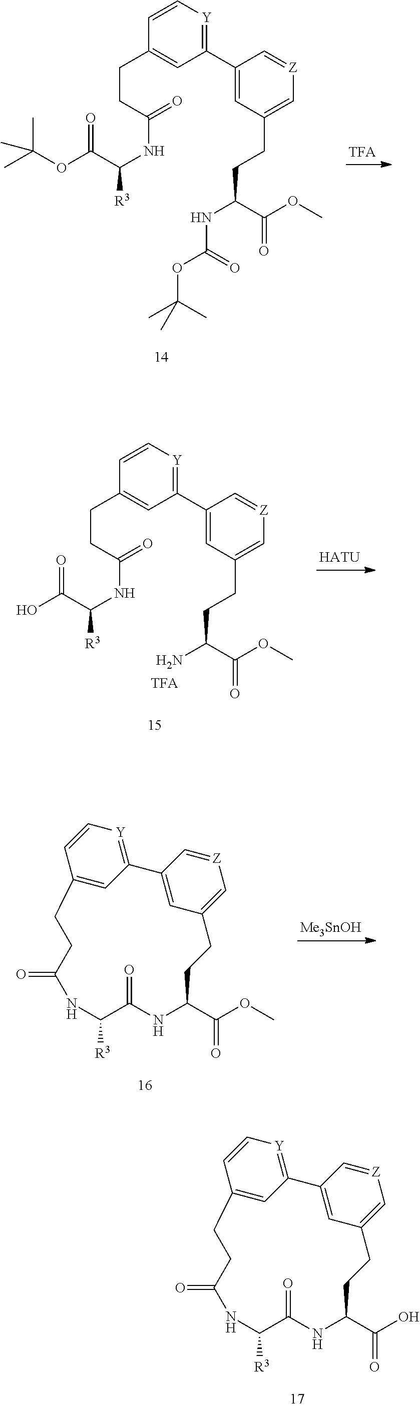

As shown in Scheme 3, the appropriately protected homophenylalanine derivative 11 can be selectively borolated using bis(pinacolato)diboron 12 under iridium catalysis (similar to methodology described in Org. Lett. 2010, 12, 3870) to provide 13 as the major regioisomer. Biaryl derivative 14 can be made by Suzuki coupling of 10 and 13. Biaryl derivative 14 can then be deprotected with trifluoroacetic acid (TFA) to reveal the amino acid 15. Compound 16 can be afforded by macrocyclization in the presence of an activating reagent such as HATU under conditions of high dilution. Ester hydrolysis using trimethyltin hydroxide (Angew. Chem. Int, Ed. 2005, 44, 1378) can give the key acid intermediate 17.

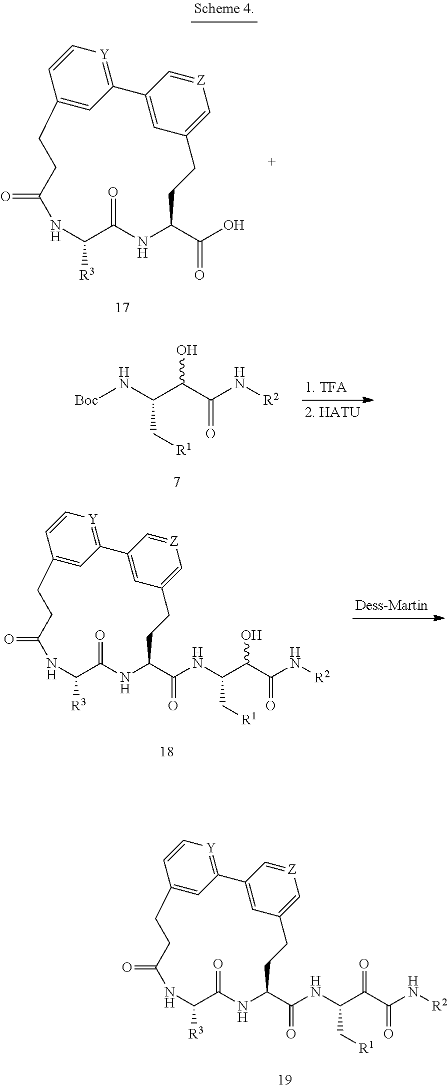

The hydroxyamide 7 can be treated with TFA as shown in Scheme 4. The free amine salt thus generated can be coupled in situ with acid 17 using an activating reagent such as HATU to afford hydroxyamide 18. Ketoamide 19 can be provided by oxidation with Dess-Martin periodinane.

### EXAMPLES

Although certain exemplary embodiments are depicted and described herein, the compounds of the present invention can be prepared using appropriate starting materials according to the methods described generally herein and/or by methods available to one of ordinary skill in the art. All reactions involving air-sensitive reagents were performed under an inert atmosphere. Reagents were used as received from commercial suppliers unless otherwise noted.

### Example 1

### (9S,12S)-12-Methyl-11,14-dioxo-10,13-diaza-tricyclo[15.3.1.1^{2,6}]docosa-1(20),2(22),3,5,17(21),18-hexaene-9-carboxylic acid ((S)-1-benzyl-2-benzylcarbamoyl-2-oxo-ethyl)-amide

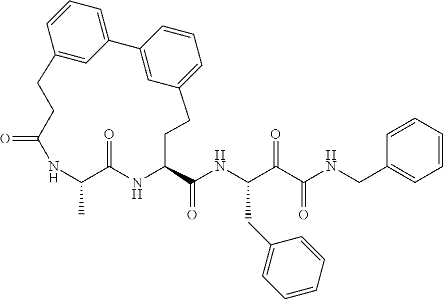 [SMILES: C[C@@H]1NC(=O)CCc2cccc(c2)-c2cccc(c2)CC[C@@H](C(=O)N[C@@H](Cc2ccccc2)C(=O)C(=O)NCc2ccccc2)NC1=O]

Step 1

To a solution of (S)-2-tert-butoxycarbonylamino-3-phenyl-propionic acid (25 g, 94.34 mmol) in DMF (250 mL) were added N,O-dimethylhydroxylamine hydrochloride (13.72 g, 141.50 mmol), HATU (37.64 g, 99.05 mmol) and N,N-diisopropylethylamine (50.70 mL, 283.01 mmol) under nitrogen atmosphere at room temperature. The reaction mixture was stirred at room temperature for 16 h then diluted with ethyl acetate (1000 mL) and washed with water (5×250 mL). The organic layer was dried and concentrated under reduced pressure. The crude residue was purified by CombiFlash column chromatography using 20% EtOAc in hexane to afford 27.5 g (94%) of (S)-1-(methoxy-methyl-carbamoyl)-2-phenyl-ethyl]-carbamic acid tert-butyl ester as colorless oil. LC/MS: (M+H)⁺=309.0.

Step 2

To a stirred solution of (S)-1-(methoxy-methyl-carbamoyl)-2-phenyl-ethyl]-carbamic acid tert-butyl ester (15 g, 48.70 mmol) in THF (180 mL) at 0° C. was added LiAlH₄ (1.0 M in THF, 57 mL, 57 mmol). The reaction mixture was stirred at 0° C. for 1 h then carefully quenched by portionwise addition of sodium sulfate decahydrate until gas evolution ceased. EtOAc was added and the reaction mixture was stirred vigorously at room temperature for 30 min and then filtered. The filtrate was dried and concentrated under reduced pressure to afford 11.0 g (91%) of ((S)-1-benzyl-2-oxo-ethyl)-carbamic acid tert-butyl ester as white solid which was used without further purification.

Step 3

To a solution of ((S)-1-benzyl-2-oxo-ethyl)-carbamic acid tert-butyl ester (7.0 g, 28.1 mmol) in DCM (80 mL) was added acetone cyanohydrin (7.16 g, 84.3 mmol) and triethylamine (2.36 mL, 16.86 mmol). The reaction was stirred at room temperature for 3 h then water was added and the organics were removed under reduced pressure. The aqueous residue was extracted with ethyl acetate and washed twice with water. The organic layer was dried and concentrated under reduced pressure. The crude residue was purified by CombiFlash column chromatography using 20% EtOAc in hexane as mobile phase to obtain 5.0 g (58%) of ((S)-1-benzyl-2-cyano-2-hydroxy-ethyl)-carbamic acid tert-butyl ester as yellow oil. LC/MS: (M+H)⁺=277.4.

Step 4

A solution of ((S)-1-benzyl-2-cyano-2-hydroxy-ethyl)-carbamic acid tert-butyl ester (5.0 g, 18.11 mmol) in 6M HCl (90 mL) was heated at 100° C. for 16 h then cooled to room temperature and concentrated under vacuum to afford 4.0 g (95%) of (S)-3-amino-2-hydroxy-4-phenyl-butyric acid hydrochloride as off yellow solid which was used without further purification. LC/MS: (M+H)⁺=196.2.

Step 5

To a solution of (S)-3-amino-2-hydroxy-4-phenyl-butyric acid hydrochloride (18.0 g, 77.9 mmol) in 1,4-dioxane (150 ml) and water (150 mL) were added sodium bicarbonate (65.45 g 779 mmol) and di-tert-butyl dicarbonate (25.48 g, 116.9 mmol). The mixture was stirred vigorously at room temperature for 16 h. The organic phase was removed under reduced pressure. The remaining heterogeneous aqueous layer was diluted with water (200 mL) and extracted with Et₂O (2×200 mL, discarded). Then the aqueous layer was brought to pH 3 by addition of aqueous 2 M HCl and extracted with EtOAc (3×400 mL). The combined extracts were dried and concentrated under reduced pressure to afford 18.0 g (78%) of (S)-3-tert-butoxycarbonylamino-2-hydroxy-4-phenyl-butyric acid as off white solid. LC/MS: (M+H)⁺296.6.

Step 6

To a stirred solution of (S)-3-tert-butoxycarbonylamino-2-hydroxy-4-phenyl-butyric acid (10.0 g, 33.89 mmol) in DMF (150 mL) were added benzylamine (4.35 g, 40.67 mmol), HATU (14.16 g, 37.28 mmol) and N,N-diisopropylethylamine (6.56 g, 50.84 mmol). The reaction mixture was stirred under nitrogen atmosphere at room temperature for 3 h then diluted with ethyl acetate (800 mL) and washed with ice cold water (2×950 mL). The organic layer was dried over sodium sulfate and concentrated under reduced pressure. The crude residue was purified by CombiFlash column chromatography using 30% EtOAc in hexane to provide 7.3 g (56%) of (S)-1-benzyl-2-benzylcarbamoyl-2-hydroxy-ethyl)-carbamic acid tert-butyl ester as white solid. LC/MS: (M+H)⁺=385.2.

Step 7

To a solution of 3-(3-bromophenyl)propanoic acid (800 mg, 3.49 mmol), (S)-tert-butyl 2-aminopropanoate hydrochloride (698 mg, 3.84 mmol), and HATU (1.46 g, 3.84 mmol) in DMF (8 ml) at 0° C. was added N,N-diisopropylethylamine (1.83 ml, 10.5 mmol). The yellow reaction mixture was stirred at room temperature overnight then quenched with water and extracted with

EtOAc (2×). The combined organics were washed with water (3×) and brine then dried over MgSO₄ and concentrated. The residue was purified by chromatography (10% to 30% EtOAc/hexanes) to afford 1.11 g (89%) of (S)-2-[3-(3-bromo-phenyl)-propionylamino]-propionic acid tert-butyl ester as a viscous colorless oil. ¹H NMR (300 MHz, CDCl₃) δ: 7.30-7.38 (m, 2H), 7.10-7.21 (m, 2H), 5.99 (d, J=6.8 Hz, 1H), 4.46 (quin, J=7.1 Hz, 1H), 2.86-3.04 (m, 2H), 2.36-2.62 (m, 2H), 1.46 (s, 9H), 1.32 (d, J=7.2 Hz, 3H).

Step 8

To a solution of (S)-2-(tert-butoxycarbonylamino)-4-phenylbutanoic acid (1.0 g, 3.58 mmol) in MeOH (20 ml) at 0° C. was added dropwise trimethylsilyldiazomethane (2.0 M in Et₂O, 3.6 ml, 7.2 mmol). Additional trimethylsilyldiazomethane (2.0 M in Et₂O) was added in 1 mL aliquots until a pale yellow color persisted. A total of 9 mL (˜5 eq) of reagent was added. The reaction was quenched with a few drops of acetic acid where upon the solution became colorless. The mixture was concentrated. The residue was absorbed on silica gel and purified by chromatography (10% to 30% EtOAc/hexanes) to afford 1.03 g (98%) of (S)-2-tert-butoxycarbonylamino-4-phenyl-butyric acid methyl ester as a colorless oil. ¹H NMR (300 MHz, CDCl₃) δ: 7.27-7.33 (m, 2H), 7.15-7.24 (m, 3H), 5.07 (d, J=7.9 Hz, 1H), 4.31-4.44 (m, 1H), 3.73 (s, 3H), 2.64-2.73 (m, 2H), 2.09-2.25 (m, 1H), 1.88-2.03 (m, 1H), 1.46 (s, 9H).

Step 9

In a 35 mL pressure tube were placed bis(pinacolato)diboron (952 mg, 3.75 mmol), 4,4′-di-tert-butyl-2,2′-bipyridine (37 mg, 0.14 mmol), and [Ir(OMe)COD)]₂ (45 mg, 0.07 mmol). A solution of (S)-2-tert-butoxycarbonylamino-4-phenyl-butyric acid methyl ester (1.0 g, 3.41 mmol) in hexanes (16 ml) was added. The tube was purged with nitrogen then sealed and heated at 65° C. for 16 h. The dark maroon reaction was cooled to room temperature, diluted with CH₂Cl₂, transferred a flask and concentrated. The residue was absorbed on silica gel and purified by chromatography (10% to 25% EtOAc/hexanes) to isolate 900 mg of a viscous colorless oil. NMR analysis indicated an approximate 3:1:1 mixture of (S)-2-tert-butoxycarbonylamino-4-[3-(4,4,5,5-tetramethyl-[1,3,2]dioxaborolan-2-yl)-phenyl]-butyric acid methyl ester: ¹H NMR (300 MHz, CDCl₃) δ: 7.66 (d, J=6.9 Hz, 1H), 7.64 (s, 1H), 7.31-7.33 (m, 1H), 7.21 (d, J=8.1 Hz, 1H), 5.08 (d, J=7.6 Hz, 1H), 4.38 (br. s., 1H), 3.75 (s, 3H), 2.65-2.74 (m, 2H), 2.11-2.23 (m, 1H), 1.89-2.02 (m, 1H), 1.48 (s, 9H), 1.37 (s, 12H); (S)-2-tert-butoxycarbonylamino-4-[4-(4,4,5,5-tetramethyl-[1,3,2]dioxaborolan-2-yl)-phenyl]-butyric acid methyl ester: ¹H NMR (300 MHz, CDCl₃) δ: 7.75 (d, J=7.8 Hz, 2H), 7.31-7.35 (m, 2H), 5.08 (d, J=7.6 Hz, 1H), 4.38 (br. s., 1H), 3.75 (s, 3H), 2.65-2.74 (m, 2H), 2.11-2.23 (m, 1H), 1.89-2.02 (m, 1H), 1.48 (s, 9H), 1.36 (s, 12H); and (S)-4-[3,5-bis-(4,4,5,5-tetramethyl-[1,3,2]dioxaborolan-2-yl)-phenyl]-2-tert-butoxycarbonylamino-butyric acid methyl ester: ¹H NMR (300 MHz, CDCl₃) δ: 8.14 (s, 1H), 7.73 (s, 2H), 5.08 (d, J=7.6 Hz, 1H), 4.38 (br. s., 1H), 3.74 (s, 3H), 2.65-2.74 (m, 2H), 2.11-2.23 (m, 1H), 1.89-2.02 (m, 1H), 1.47 (s, 9H), 1.36 (s, 24H).

Step 10

To a solution of (S)-2-[3-(3-bromo-phenyl)-propionylamino]-propionic acid tert-butyl ester (918 mg, 2.58 mmol) and (S)-2-tert-butoxycarbonylamino-4-[3-(4,4,5,5-tetramethyl-[1,3,2]dioxaborolan-2-yl)-phenyl]-butyric acid methyl ester (major component of mixture from Step 3, 900 mg, 2.15 mmol) in 1,4-dioxane (12 ml) were added Pd(PPh₃)₄ (124 mg, 0.11 mmol) and 2.0 M aqueous sodium carbonate (3.2 ml, 6.4 mmol). The biphasic mixture was stirred at 90° C. for 3.5 h then cooled to room temperature, quenched with water and extracted with EtOAc (2×). The combined organics were dried over MgSO₄ and concentrated. The residue was absorbed on silica gel and purified by chromatography (20% to 40% EtOAc/hexanes) to isolate 647 mg (53%) of a viscous colorless oil. NMR analysis indicated an approximate 3:1 mixture of (S)-2-tert-butoxycarbonylamino-4-{3′-[2-((S)-1-tert-butoxycarbonyl-ethylcarbamoyl)-ethyl]-biphenyl-3-yl}-butyric acid methyl ester and (S)-2-tert-butoxycarbonylamino-4-{3′-[2-((S)-1-tert-butoxycarbonyl-ethylcarbamoyl)-ethyl]-biphenyl-4-yl}-butyric acid methyl ester. LC/MS: (M+Na)⁺=591.

Step 11

To a solution of (S)-2-tert-butoxycarbonylamino-4-{3′-[2-((S)-1-tert-butoxycarbonyl-ethylcarbamoyl)-ethyl]-biphenyl-3-yl}-butyric acid methyl ester (major component of mixture from Step 4, 647 mg, 1.14 mmol) in dichloromethane (8 ml) was added TFA (2 ml, 26.0 mmol). The light yellow reaction mixture was stirred at room temperature for 2.5 h. Additional TFA (2 ml) was added and stirring continued for 2.5 h then the mixture was concentrated and chased with dichloromethane (3×). The residue was dissolved in dichloromethane (200 ml) and DMF (50 ml). Then added HATU (519 mg, 1.37 mmol) followed by N,N-diisopropylethylamine (1.6 ml, 9.1 mmol). The yellow reaction mixture was stirred at room temperature for 96 h. The dichloromethane was removed under reduced pressure. Water (200 mL) was added with ice cooling. The aqueous layer was extracted with Et₂O (200 mL) and then with EtOAc (200 mL). The combined organics were washed with water (4×) and brine then dried over MgSO₄ and concentrated. The crude residue was absorbed on silica gel and purified by chromatography first with 50% to 100% EtOAc/hexanes then with 0% to 2% MeOH/CH₂Cl₂ to afford 83 mg (19%) of (9S,12S)-12-methyl-11,14-dioxo-10,13-diaza-tricyclo[15.3.1.1^{2,6}]docosa-1(20),2(22),3,5,17(21),18-hexaene-9-carboxylic acid methyl ester as a white solid. LC/MS: (M+H)⁺=395; ¹H NMR (300 MHz, CDCl₃) δ: 7.27-7.47 (m, 5H), 7.04-7.11 (m, 3H), 6.64 (d, J=7.9 Hz, 1H), 6.40 (d, J=8.3 Hz, 1H), 4.86-5.00 (m, 1H), 4.18 (ddd, J=11.4, 7.6, 4.0 Hz, 1H), 3.68 (s, 3H), 3.20 (ddd, J=13.8, 10.8, 2.6 Hz, 1H), 2.86 (ddd, J=13.8, 8.5, 2.6 Hz, 1H), 2.66 (dd, J=8.5, 4.5 Hz, 2H), 2.55-2.63 (m, 1H), 2.34-2.46 (m, 1H), 2.15-2.30 (m, 1H), 1.94-2.08 (m, 1H), 1.40 (d, J=6.8 Hz, 3H).

Step 12

To a solution of (9S,12S)-12-methyl-11,14-dioxo-10,13-diaza-tricyclo[15.3.1.1^{2,6}]docosa-1(20),2(22),3,5,17(21),18-hexaene-9-carboxylic acid methyl ester (80 mg, 0.20 mmol) in 1,2-dichloroethane (3 ml) was added trimethyltin hydroxide (183 mg, 1.01 mmol). The cloudy reaction mixture was stirred at 80° C. for 6 h then cooled to room temperature and concentrated.

The residue was partitioned between EtOAc and 1.0M HCl. The aqueous layer was extracted with EtOAc. The combined organics were washed with 1.0M HCl (5×) and dried over MgSO₄ then concentrated and dried under high vacuum to afford (9S,12S)-12-methyl-11,14-dioxo-10,13-diaza-tricyclo[15.3.1.1^{2,6}]docosa-1(20),2(22),3,5,17(21),18-hexaene-9-carboxylic acid as a white semisolid which was used without further purification.

Step 13

To a solution of ((S)-1-benzyl-2-benzylcarbamoyl-2-hydroxy-ethyl)-carbamic acid tert-butyl ester (93 mg, 0.24 mmol) in dichloromethane (2 ml) was added TFA (0.50 ml, 6.5 mmol). The reaction mixture was stirred at room temperature for 3 h then concentrated, chased with dichloromethane (2×) and dried under high vacuum to obtain a pale yellow oil. To the oil was added a solution of (9S,12S)-12-methyl-11,14-dioxo-10,13-diaza-tricyclo[15.3.1.1^{2,6}]docosa-1(20),2(22),3,5,17(21),18-hexaene-9-carboxylic acid (crude from Step 6, 77 mg, 0.20 mmol) in DMF (2 ml). Then added HATU (85 mg, 0.22 mmol) and N,N-diisopropylethylamine (0.18 ml, 1.01 mmol). The yellow reaction mixture was stirred at room temperature overnight then quenched with water. The resultant precipitate was collected via filtration, washed with water, and dried by suction then under high vacuum to afford 110 mg (84%) of (9S,12S)-12-methyl-11,14-dioxo-10,13-diaza-tricyclo[15.3.1.1^{2,6}]docosa-1(20),2(22),3,5,17(21),18-hexaene-9-carboxylic acid ((S)-1-benzyl-2-benzylcarbamoyl-2-hydroxy-ethyl)-amide as an off-white solid and a mixture of epimers. LC/MS: (M+Na)⁺=669.

Step 14

To a slightly cloudy solution of (9S,12S)-12-methyl-11,14-dioxo-10,13-diaza-tricyclo[15.3.1.1^{2,6}]docosa-1(20),2(22),3,5,17(21),18-hexaene-9-carboxylic acid ((S)-1-benzyl-2-benzylcarbamoyl-2-hydroxy-ethyl)-amide (105 mg, 0.16 mmol) in dichloromethane (8 ml) was added Dess-Martin periodinane (103 mg, 0.24 mmol). The reaction mixture was stirred at room temperature for 2 h during which time a thick precipitate had formed. Quenched by addition of sat'd NaHCO₃ (5 ml) and 10% Na₂S₂O₃ (5 ml). The biphasic mixture was stirred vigorously for 20 min then the layers were separated. The aqueous layer was extracted with dichloromethane (2×). The combined organic layers were washed with sat'd NaHCO₃ then dried over MgSO₄ and concentrated to a light yellow solid. Trituration with MeOH/Et₂O provided 38 mg (36%) of (9S,12S)-12-methyl-11,14-dioxo-10,13-diaza-tricyclo[15.3.1.1^{2,6}]docosa-1(20),2(22),3,5,17(21),18-hexaene-9-carboxylic acid ((S)-1-benzyl-2-benzylcarbamoyl-2-oxo-ethyl)-amide as a white solid. LC/MS: (M+Na)⁺=667; ¹H NMR (400 MHz, DMSO-d₆) δ: 9.20 (t, J=6.4 Hz, 1H), 8.52 (d, J=8.1 Hz, 1H), 8.19 (d, J=9.1 Hz, 1H), 8.01 (d, J=6.8 Hz, 1H), 7.16-7.52 (m, 14H), 7.08-7.15 (m, 4H), 5.12-5.19 (m, 1H), 4.70-4.81 (m, 1H), 4.31 (d, J=6.4 Hz, 2H), 3.79-3.88 (m, 1H), 3.02-3.09 (m, 1H), 3.00 (dd, J=9.3, 6.1 Hz, 1H), 2.78-2.88 (m, 2H), 2.75 (d, J=6.3 Hz, 2H), 2.53-2.59 (m, 1H), 2.15 (ddd, J=12.9, 9.5, 2.9 Hz, 1H), 1.83-1.92 (m, 2H), 1.16 (d, J=7.1 Hz, 3H).

### Example 2

The following compounds are made by a procedure analogous to that described in Example 1:

| Compound | Procedure |
|---|---|
| 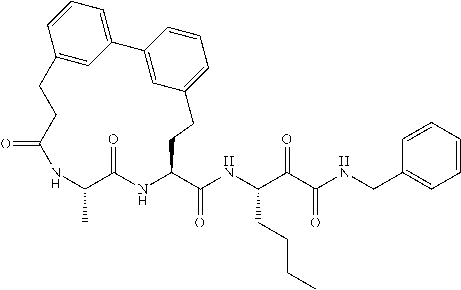 [SMILES: CCCC[C@H](NC(=O)[C@@H]1CCc2cccc(c2)-c2cccc(c2)CCC(=O)N[C@@H](C)C(=O)N1)C(=O)C(=O)NCc1ccccc1] | Analogous to Example 1 by substituting (S)-2-tert-butoxycarbonylamino-hexanoic acid for (S)-2-tert-butoxycarbonylamino-3- phenyl-propionic acid in Step 1. |
| 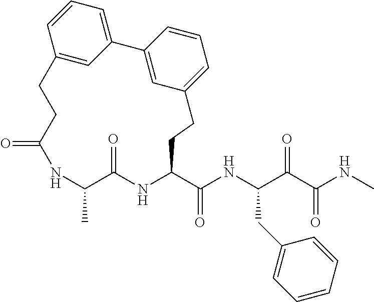 [SMILES: CNC(=O)C(=O)[C@H](Cc1ccccc1)NC(=O)[C@@H]1CCc2cccc(c2)-c2cccc(c2)CCC(=O)N[C@@H](C)C(=O)N1] | Analogous to Example 1 by substituting methylamine for benzylamine in Step 6. |
| 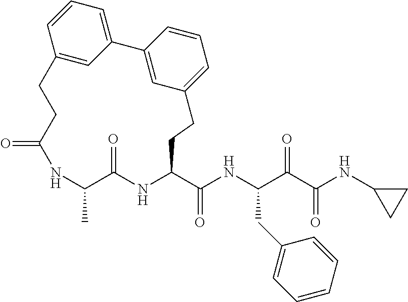 [SMILES: C[C@@H]1NC(=O)CCc2cccc(c2)-c2cccc(c2)CC[C@@H](C(=O)N[C@@H](Cc2ccccc2)C(=O)C(=O)NC2CC2)NC1=O] | Analogous to Example 1 by substituting cyclopropylamine for benzylamine in Step 6. |
| 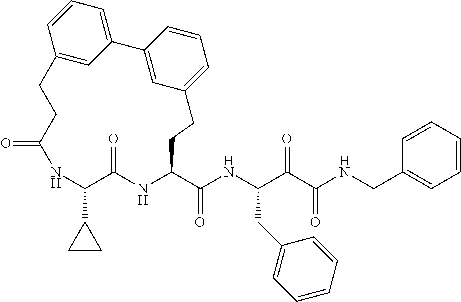 [SMILES: O=C1CCc2cccc(c2)-c2cccc(c2)CC[C@@H](C(=O)N[C@@H](Cc2ccccc2)C(=O)C(=O)NCc2ccccc2)NC(=O)[C@H](C2CC2)N1] | Analogous to Example 1 by substituting (S)-2- amino-2-cyclopropyl-acetic acid tert- butyl ester for (S)-tert-butyl 2- aminopropanoate hydrochloride in Step 7. |
| 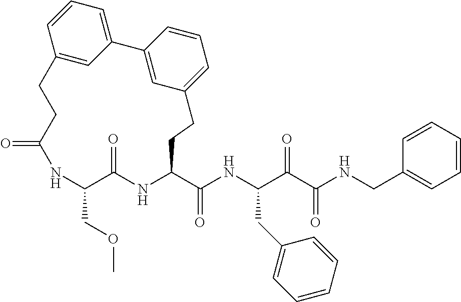 [SMILES: COC[C@@H]1NC(=O)CCc2cccc(c2)-c2cccc(c2)CC[C@@H](C(=O)N[C@@H](Cc2ccccc2)C(=O)C(=O)NCc2ccccc2)NC1=O] | Analogous to Example 1 by substituting (S)-2-amino-3-methoxy-propionic acid tert- butyl ester for (S)-tert-butyl 2- aminopropanoate hydrochloride in Step 7. |
| 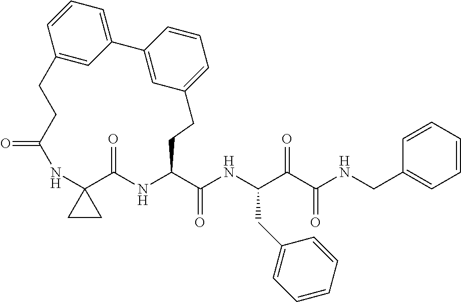 [SMILES: O=C1CCc2cccc(c2)-c2cccc(c2)CC[C@@H](C(=O)N[C@@H](Cc2ccccc2)C(=O)C(=O)NCc2ccccc2)NC(=O)C2(CC2)N1] | Analogous to Example 1 by substituting 1-amino-cyclopropanecarboxylic acid tert- butyl ester for (S)-tert-butyl 2- aminopropanoate hydrochloride in Step 7. |
| 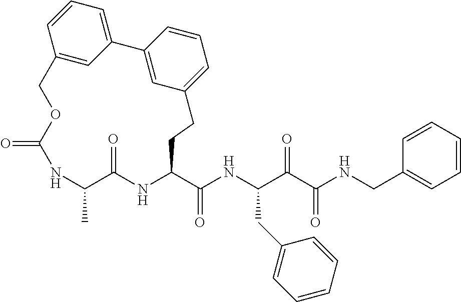 [SMILES: C[C@@H]1NC(=O)OCc2cccc(c2)-c2cccc(c2)CC[C@@H](C(=O)N[C@@H](Cc2ccccc2)C(=O)C(=O)NCc2ccccc2)NC1=O] | Analogous to Example 1 by substituting 3-bromobenzyl chloroformate for 3-(3- bromophenyl)propanoic acid, dichloromethane for DMF and omitting HATU in Step 7. |
| 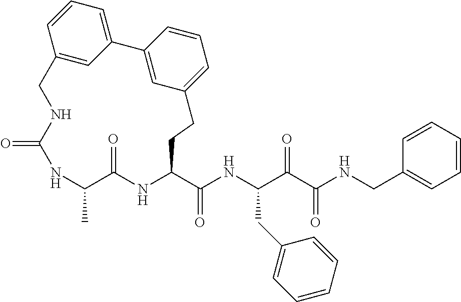 [SMILES: C[C@@H]1NC(=O)NCc2cccc(c2)-c2cccc(c2)CC[C@@H](C(=O)N[C@@H](Cc2ccccc2)C(=O)C(=O)NCc2ccccc2)NC1=O] | Analogous to Example 1 by substituting 3-bromobenzyl carbamic chloride for 3-(3- bromophenyl)propanoic acid, dichloromethane for DMF and omitting HATU in Step 7. |
| 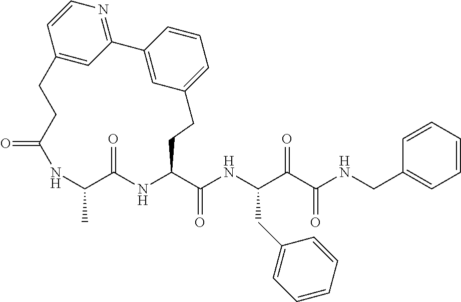 [SMILES: C[C@@H]1NC(=O)CCc2ccnc(c2)-c2cccc(c2)CC[C@@H](C(=O)N[C@@H](Cc2ccccc2)C(=O)C(=O)NCc2ccccc2)NC1=O] | Analogous to Example 1 by substituting 3-(2-bromo-pyridin-4-yl)-propanoic acid for 3-(3-bromophenyl)propanoic acid in Step 7. |
| 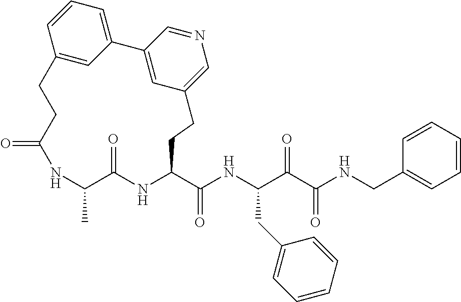 [SMILES: C[C@@H]1NC(=O)CCc2cccc(c2)-c2cncc(c2)CC[C@@H](C(=O)N[C@@H](Cc2ccccc2)C(=O)C(=O)NCc2ccccc2)NC1=O] | Analogous to Example 1 by substituting (S)-2-tert-butoxycarbonylamino-4-pyridin-3- yl-butyric acid methyl ester for (S)-2-tert- butoxycarbonylamino-4-phenyl-butyric acid methyl ester in Step 9. |

### Example 3

### Assay Protocols and Results

Cell-Based Proteasome Activity/Selectivity Assay

The Cell-Based Proteasome subunit activity/selectivity assay was a panel of 5 fluorogenic assays that independently measured the activity of β5c or β 5i (chymotrypsin-like activity), β2c/2i (trypsin-like), and β 1c or β 1i (caspase-like) protease activity associated with the proteasome complex in cultured cells. Specifically, the following substrates were used for respective subunit activities: β 1i: (PAL)₂Rh110, β 1c: (LLE)₂ Rh110, β2c/2i: (KQL)₂Rh110, β 5c: (WLA)₂Rh110, β 5i: (ANW)₂Rh110. The following procedure was followed:

Cell preparation: Plated 25 μl of Ramos cells (2×10⁶/ml in DPBS) into half area plate (PerkinElmer Cat 6005569) to final 5×10⁴ cells/well. Added 0.5 μl of 100×4-fold serial diluted test compounds or DMSO to each well. Highest concentration of compound tested was 20 thus compound serial dilution started from 200 mM. Incubated for 30 minutes at 37° C. Then equilibrated at room temperature for 15 minutes. Added 25 μl of 2× reaction mix consisting of 0.025% digitonin, 20 μM of each substrates and 0.5M sucrose in DPBS. Shaked for one minute @ 700 rpm. Incubated for 120 mM at room temperature. Then read the plates with an Envision multilabel plate reader (PerkinElmer) with 500 nm excitation/519 nm emission.

Modified PBMC Proteasome Activity Assay

This cell-based proteasome activity assay was similar to previous Ramos cell-based assay as of the substrates, but using human PBMCs in the context of complete RPMI with 10% FBS as reaction buffer. This assay was designed to assess the level of cellular penetration of test compounds in primary human cells. The following procedure was followed: Fresh isolated

PBMC from healthy donor were plated at 1×10⁵ cells/well in 100 μl of complete RPMI with 10% FBS in V bottom 96 plates. Added 1 μl of 100×4-fold serial diluted compounds/well and incubated for 1 hr. The highest compound concentration tested was 20 μM (100× working stock start with 2 mM). Spun down the cells @ 2000 rpm for 5 min. Removed all supernatant. Then resuspended the cells in 25 μl DPBS and transferred the cells to a fresh half-area plate (PerkinElmer Cat 6005569). In the final reaction volume was 50 μl, including 25 μl cell suspension, 0.5 μl 100× inhibitor or DMSO, 25 μl substrate mix containing 0.025% digitonin, 20 uM substrate (Substrate: (PAL)2Rh110, (LLE)₂ Rh110, (KQL)2Rh110, (WLA)₂Rh110, or (ANW)₂Rh110)/in 10% FBS and 0.5M sucrose mixture. Shaked for one minute (@ 700 rpm). Incubated for 2 hrs, then read the plates with Envision plate reader using 500 nm excitation/519 nm emission.

PBMC IP-10 Assay

PBMCs were isolated from whole blood as follows: Blood was collected in a sterile environment in heparinized tubes. Blood was diluted with an equal volume PBS/2% FCS and 30 ml of this mixture was added to ACCUSPIN tubes containing 15 ml Histopaque-1077 already centrifuged at 800 g for 30 seconds and warmed up at room temperature. The tubes were then centrifuged at 800 g for 20 minutes at room temperature with no brake. The mononuclear band, just above the polyethylene frit, was removed by Pasteur pipet. These mononuclear cells were washed three times with sterile PBS, counted, and resuspended in RPMI 1640 supplemented with 10% heat inactivated fetal calf serum, 10 mM HEPES, 1 mM sodium pyruvate, penicillin (50 U/ml), streptomycin (50 μg/ml) and glutamine (2 mM) to approximately 1.5×10⁶/ml. Approximately 2×10⁵ cells/well were plated in 96 well tissue culture plates (BD Falcon 353072), and preincubated 60 ml/37° C. with a titration of compounds, in a final concentration of 1% DMSO. Cells were then stimulated with CpG Type A (Invivogen, Cat # tlrl-2216; ODN 2216) at a final concentration of 2.5 μM. Cells were incubated overnight, and supernatants were removed. PBMC viability of cells remaining in the well was measured with ATPlite luminescence assay (Perkin-Elmer) per the manufacturer's instructions. Luminescence was measured on the Perkin-Elmer Envision, using the luminescence filter. IP 10 level was measured with CXCL10/IP10 AlphaLISA kit (Perkin-Elmer) per the manufacturer's instructions, except halving all volumes. Fluorescence was measured on the Envision Multilabel plate reader, using the AlphaScreen standard settings.

Results:

The results of the above assays for representative compounds of the invention are provided in Table 1 below, wherein the IC50 and EC50 activity values are in μM:

TABLE 1
It is to be understood that the invention is not limited
to the particular embodiments of the invention described above,
as variations of the particular embodiments may be made and
still fall within the scope of the appended claims.

| | Ic50 | Ic50 | Ic50 | Ic50 | Ic50 | |
|---|---|---|---|---|---|---|
| | ramos: | ramos: | ramos: | ramos: | ramos: | |
| Example | ac-(anw)2- | rh110- | rh110- | rh110- | rh110- | |
| No. | r110 | (wla)2 | (kql)2 | (pal)2 | (lle)2 | Ec50 |
| 1 | 0.019 | 0.353 | 20 | 1.975 | 20 | 0.347 |

1. A compound of formula (I): 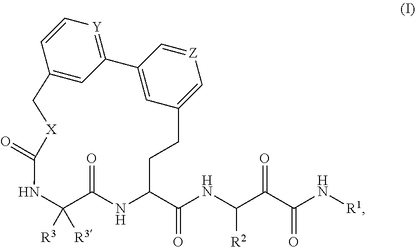
wherein:
X is CH₂, 0 or NH;
Y is CH or N;
Z is CH or N;
R¹ is C₁₋₇ alkyl, C₃₋₈ cycloalkyl or —CH₂-phenyl;
R² is C₁₋₇ alkyl or —CH₂-phenyl; and
one of R³ or R^{3′} is hydrogen and the other is C₃₋₈ cycloalkyl, unsubstituted C₁₋₇ alkyl or C₁₋₇ alkyl substituted with C₁₋₇ alkoxy, or
R³ and R^{3′}, together with the carbon atom to which they are attached, combine to form a C₃₋₈ cycloalkyl moiety,
or a pharmaceutically acceptable salt thereof.

2. The compound according to claim 1, wherein X is CH₂.

3. The compound according to claim 1, wherein X is O or NH.

4. The compound according to claim 1, wherein Y is CH.

5. The compound according to claim 1, wherein Y is N.

6. The compound according to claim 1, wherein Z is CH.

7. The compound according to claim 1, wherein Z is N.

8. The compound according to claim 1, wherein X is CH₂, Y and Z are CH.

9. The compound according to claim 1, wherein R¹ is methyl, —CH₂-phenyl or cyclopropyl.

10. The compound according to claim 1, wherein R′ is —CH₂-phenyl.

11. The compound according to claim 1, wherein R² is butyl or —CH₂-phenyl.

12. The compound according to claim 1, wherein one of R³ or R^{3′} is hydrogen and the other is cyclopropyl, methyl, or —CH₂OCH₃.

13. The compound according to claim 1, wherein R³ is methyl and R^{3′} is hydrogen.

14. The compound according to claim 1, wherein R³ and R^{3′}, together with the carbon atom to which they are attached, combine to form a cyclopropyl moiety.

15. The compound according to claim 1, wherein said compound is:

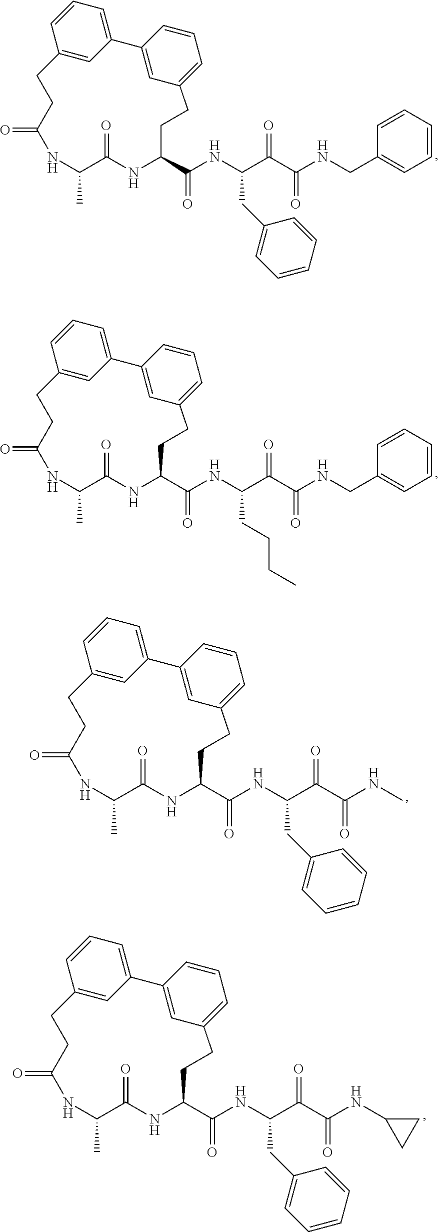 [SMILES: C[C@@H]1NC(=O)CCc2cccc(c2)-c2cccc(c2)CC[C@@H](C(=O)N[C@@H](Cc2ccccc2)C(=O)C(=O)NCc2ccccc2)NC1=O ; CCCC[C@H](NC(=O)[C@@H]1CCc2cccc(c2)-c2cccc(c2)CCC(=O)N[C@@H](C)C(=O)N1)C(=O)C(=O)NCc1ccccc1 ; CNC(=O)C(=O)[C@H](Cc1ccccc1)NC(=O)[C@@H]1CCc2cccc(c2)-c2cccc(c2)CCC(=O)N[C@@H](C)C(=O)N1 ; C[C@@H]1NC(=O)CCc2cccc(c2)-c2cccc(c2)CC[C@@H](C(=O)N[C@@H](Cc2ccccc2)C(=O)C(=O)NC2CC2)NC1=O]

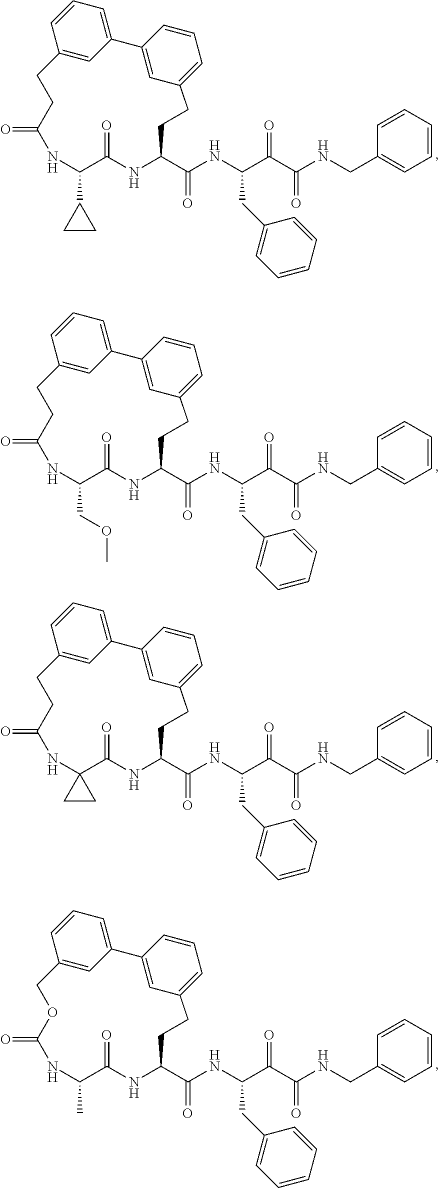 [SMILES: O=C1CCc2cccc(c2)-c2cccc(c2)CC[C@@H](C(=O)N[C@@H](Cc2ccccc2)C(=O)C(=O)NCc2ccccc2)NC(=O)[C@H](C2CC2)N1 ; COC[C@@H]1NC(=O)CCc2cccc(c2)-c2cccc(c2)CC[C@@H](C(=O)N[C@@H](Cc2ccccc2)C(=O)C(=O)NCc2ccccc2)NC1=O ; O=C1CCc2cccc(c2)-c2cccc(c2)CC[C@@H](C(=O)N[C@@H](Cc2ccccc2)C(=O)C(=O)NCc2ccccc2)NC(=O)C2(CC2)N1 ; C[C@@H]1NC(=O)OCc2cccc(c2)-c2cccc(c2)CC[C@@H](C(=O)N[C@@H](Cc2ccccc2)C(=O)C(=O)NCc2ccccc2)NC1=O]

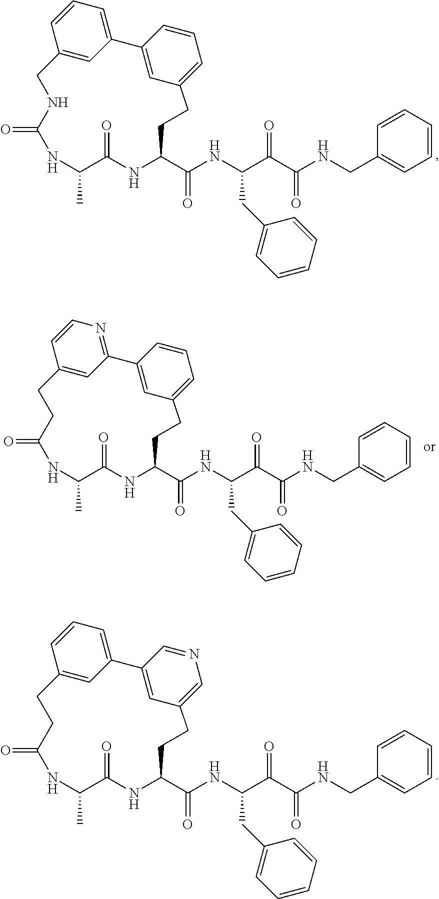 [SMILES: C[C@@H]1NC(=O)NCc2cccc(c2)-c2cccc(c2)CC[C@@H](C(=O)N[C@@H](Cc2ccccc2)C(=O)C(=O)NCc2ccccc2)NC1=O ; C[C@@H]1NC(=O)CCc2ccnc(c2)-c2cccc(c2)CC[C@@H](C(=O)N[C@@H](Cc2ccccc2)C(=O)C(=O)NCc2ccccc2)NC1=O ; C[C@@H]1NC(=O)CCc2cccc(c2)-c2cncc(c2)CC[C@@H](C(=O)N[C@@H](Cc2ccccc2)C(=O)C(=O)NCc2ccccc2)NC1=O]

16. The compound according to claim 1, wherein said compound is:

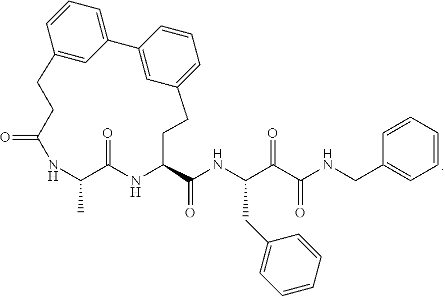 [SMILES: C[C@@H]1NC(=O)CCc2cccc(c2)-c2cccc(c2)CC[C@@H](C(=O)N[C@@H](Cc2ccccc2)C(=O)C(=O)NCc2ccccc2)NC1=O]

17. A pharmaceutical composition, comprising a therapeutically effective amount of a compound according to claim 1 and a pharmaceutically acceptable carrier.

18-21. (canceled)

22. A method for treating an inflammatory disease or disorder selected from rheumatoid arthritis, lupus and irritable bowel disease, comprising the step of administering a therapeutically effective amount of a compound according to claim 1 to a subject in need thereof.

23. (canceled)
